(19) United States
(12) Patent Application Publication
(10) Pub. No.: US 2024/0325476 A1
(43) Pub. Date: Oct. 3, 2024

(54) METHOD FOR ALLEVIATING DRY EYE DISEASE

(71) Applicant: BioRay Biotech. Co., Ltd., Pingtung County (TW)

(72) Inventors: Ho-Shin HUANG, Pingtung County (TW); Chun-Mei LU, Pingtung County (TW); Ting-Yuan HSU, Pingtung County (TW); Bo-Yie CHEN, Pingtung County (TW)

(21) Appl. No.: 18/489,064

(22) Filed: Oct. 18, 2023

Related U.S. Application Data
(60) Provisional application No. 63/455,425, filed on Mar. 29, 2023.

(51) Int. Cl. A61K 36/068; A61P 27/02

(52) U.S. Cl. CPC A61K 36/068; A61P 27/02; A61K 2236/33

(57) ABSTRACT

A method for alleviating dry eye disease includes administering to a subject in need thereof a pharmaceutical composition containing an ethanol-extracted product of fruiting bodies of Cordyceps militaris, and the dry eye disease may be caused by a factor selected from the group consisting of a sleep disorder, aging, Sjögren's syndrome, a particulate matter (PM), and combinations thereof.

### CROSS-REFERENCE TO RELATED APPLICATION

This application claims the benefit of U.S. Provisional Patent Application No. 63/455,425, filed on Mar. 29, 2023, and incorporated by reference herein in its entirety.

### FIELD

The disclosure relates to a method for alleviating dry eye disease using a pharmaceutical composition containing an ethanol-extracted product of fruiting bodies of Cordyceps militaris.

### BACKGROUND

Dry eye syndrome (DES), also known as keratoconjunctivitis sicca (KCS), is caused by insufficient tear production and excessive tear evaporation in an eye of a human, and the main symptoms thereof are dryness, redness, itching and pain in the eye. In severe cases of DES, visual disturbance and ocular surface damage may even occur.

Treatment for DES in clinic typically involves utilization of cyclosporine, non-steroidal anti-inflammatory drugs (NSAIDs), or steroidal anti-inflammatory drugs (SAIDs). However, the aforesaid drugs not only fail to achieve a desired therapeutic effect but also may give rise to serious side effects or adverse effects. As a result, those skilled in the art strive to find an active component derived from a natural source for treatment of DES without causing undesirable side effects.

Cordyceps militaris, previously known as Clavaria militaris, is an entomopathogenic fungus from the genus Cordyceps in the family Cordycipitaceae which usually parasitizes the larvae or pupae of insects of the Lepidoptera order, and has one or more fruiting bodies growing from a head or a node of its host.

Cordyceps militaris has been used as traditional Chinese medicine (TCM), and the fruiting bodies of Cordyceps militaris are the most frequently utilized portion. It has been reported that Cordyceps militaris has anti-oxidant, anti-cancer, anti-inflammatory, and immunomodulatory activities. For instance, as reported in Lin T. Y. et al. (2017), J. Food Nutr. Res.; 5(5): 320-330, an extract of a fermentation mycelium of Cordyceps cicadae was capable of alleviating benzalkonium chloride (BAC)-induced dry eye syndrome in Institute of Cancer Research (ICR) mice.

### SUMMARY

Therefore, an object of the disclosure is to provide a method for alleviating dry eye disease, which can alleviate at least one of the drawbacks of the prior art, and which includes administering to a subject in need thereof a pharmaceutical composition containing an ethanol-extracted product of fruiting bodies of Cordyceps militaris.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the wetted length determined in each group of Example 1, infra, in which the symbol “**” represents p<0.01 (compared with the pathological control group), and the symbol “##” represents p<0.01 (compared with the comparative group).

FIG. 2 shows the tear film break-up time determined in each group of Example 1, infra, in which the symbol “**” represents p<0.01 (compared with the pathological control group), and the symbol “##” represents p<0.01 (compared with the comparative group).

FIG. 3 shows the concentric rings observed on a surface of a tear film of the mice in each group of Example 1, infra.

FIG. 4 shows the corneal staining score determined in each group of Example 1, in which the symbol “**” represents p<0.01 (compared with the pathological control group), and the symbol “##” represents p<0.01 (compared with the comparative group).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

While conducting experiments, the applicant has found that an ethanol-extracted product of fruiting bodies of Cordyceps militaris can effectively alleviate dry eye disease as well as related pathological signs thereof, and the efficacy of the ethanol-extracted product of fruiting bodies of Cordyceps militaris is significantly better than that of an ethanol-extracted product of fruiting bodies of Cordyceps cicadae. Therefore, the ethanol-extracted product of fruiting bodies of Cordyceps militaris is believed to have a high potential in alleviating dry eye disease.

Accordingly, the disclosure provides a method for alleviating dry eye disease, which includes administering to a subject in need thereof a pharmaceutical composition containing an ethanol-extracted product of fruiting bodies of Cordyceps militaris.

As used herein, the terms “dry eye disease”, “dry eye syndrome”, “xerophthalmia”, “keratoconjunctivitis sicca”, and “dysfunctional tear syndrome” may be interchangeably used, and are intended to cover at least one of the following forms: aqueous tear-deficient dry eye, mucin-deficient dry eye, lipid-deficient dry eye, and evaporative dry eye.

According to the disclosure, the dry eye disease may be caused by a factor selected from the group consisting of a sleep disorder (e.g., insomnia and sleep apnea syndrome), aging, Sjögren's syndrome, a particulate matter (PM) (e.g., PM_{2.5}), and combinations thereof. In certain embodiments, the dry eye disease is caused by the sleep disorder. In an exemplary embodiment, the sleep disorder is insomnia.

As used herein, the term “Cordyceps militaris” is intended to refer to the Cordyceps militaris that is commonly available to those skilled in the art (i.e., may be purchased from an herbal medicine store or any international depositary authorities recognized under the Budapest Treaty) or is collected or isolated from a natural source, and can be cultivated using technology well known to those skilled in the art. In an exemplary embodiment, the Cordyceps militaris is fruiting bodies of Cordyceps militaris obtained according to the cultivation method described in the Invention Patent Certificate No. TW I459953 B.

According to the disclosure, the extraction method for obtaining the ethanol-extracted product of fruiting bodies of Cordyceps militaris is within the expertise and routine skills of those skills in the art.

It can be understood that for achieving the best extraction results, the operating conditions of the extraction method for obtaining the ethanol-extracted product of fruiting bodies of Cordyceps militaris may vary depending on the following factors: the type of method used for preparing the fruiting bodies of Cordyceps militaris and the amount of the ethanol and the fruiting bodies of Cordyceps militaris utilized. The selection of the operating conditions is within the expertise and routine skills of those skilled in the art.

In certain embodiments, the ethanol-extracted product of fruiting bodies of Cordyceps militaris may be obtained by subjecting fresh fruiting bodies of Cordyceps militaris to an ethanol extraction treatment.

In certain embodiments, the ethanol-extracted product of fruiting bodies of Cordyceps militaris may be obtained by subjecting processed fruiting bodies of Cordyceps militaris to the ethanol extraction treatment.

In certain embodiments, the processed fruiting bodies of Cordyceps militaris may be obtained by subjecting the fresh fruiting bodies of Cordyceps militaris to a preliminary treatment selected from the group consisting of a drying treatment, a grinding treatment, a chopping treatment, a comminuting treatment, and combinations thereof. In an exemplary embodiment, the fresh fruiting bodies of Cordyceps militaris is subjected to a drying treatment and a comminuting treatment in sequence, so that the resultant processed fruiting bodies of Cordyceps militaris has a moisture content of less than 15% and a particle size of less than 0.4 mm.

According to the disclosure, the ethanol extraction treatment may be conducted using an ethanol solution having an ethanol concentration ranging from 20% to 95%. In certain embodiments, the ethanol solution may have an ethanol concentration ranging from 50% to 75%. In an exemplary embodiment, the ethanol solution has an ethanol concentration of 62.9%.

According to the disclosure, in the ethanol extraction treatment, a weight ratio of the fruiting bodies of Cordyceps militaris to the ethanol solution may range from 1:5 to 1:20. In certain embodiments, the weight ratio of the fruiting bodies of Cordyceps militaris to the ethanol solution may range from 1:7 to 1:15. In an exemplary embodiment, the weight ratio of the fruiting bodies of Cordyceps militaris to the ethanol solution is 1:10.

According to the disclosure, the ethanol extraction treatment may be carried out at a temperature ranging from 45° C. to 80° C. In an exemplary embodiment, the ethanol extraction treatment is carried out at 65° C.

According to the disclosure, the ethanol-extracted product of fruiting bodies of Cordyceps militaris is obtained according to the method described in the Invention Patent Certificate No. TW I772201 B.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

According to the disclosure, the pharmaceutical composition may be formulated into a dosage form suitable for oral administration using techniques well known to those skilled in the art.

According to the disclosure, the pharmaceutical composition may further include a pharmaceutically acceptable carrier widely employed in the art of drug-manufacturing. For instance, the pharmaceutically acceptable carrier may include one or more of the following agents: solvents (e.g., a sterile water), buffers (e.g., an ophthalmic balanced salt solution, phosphate-buffered saline (PBS), Ringer's solution and Hank's solution), emulsifiers, suspending agents, decomposers, pH adjusting agents, stabilizing agents, chelating agents, preservatives, diluents, absorption delaying agents, liposomes, and the like. The choice and amount of the aforesaid agents are within the expertise and routine skills of those skilled in the art.

According to the disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

As used herein, the term “administration” or “administering” can be interchangeably used, and means introducing, providing or delivering the abovementioned pharmaceutical composition to a subject showing condition(s) or symptom(s) of dry eye disease by any suitable routes to perform its intended function.

As used herein, the term “subject” refers to any animal of interest, such as humans, monkeys, cows, sheep, horses, pigs, goats, dogs, cats, mice, and rats. In an exemplary embodiment, the subject is a human.

The dose and frequency of administration of the pharmaceutical composition of the disclosure may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are intended solely for the purpose of illustration and should not be construed as limiting the present disclosure in practice.

### EXAMPLES

### General Experimental Procedures:

### 1. Statistical Analysis:

The experimental data obtained in all the test groups were statistically analyzed using Statistical Package for the Social Science (SPSS) Statistics software (Version 22.0), and are expressed as mean±standard error of the mean (SEM). The differences between the test groups were analyzed using Kruskal-Wallis test followed by Mann-Whitney U test. Statistical significance is indicated by p<0.05.

### Example 1. Evaluation of the Efficacy of Ethanol-Extracted Product of Fruiting Bodies of Cordyceps militaris in Alleviating Dry Eye Disease (DED)

### Experimental Materials:

1. Preparation of Ethanol-Extracted Product of Fruiting Bodies of Cordyceps militaris:

The ethanol-extracted product of fruiting bodies of Cordyceps militaris was prepared according to the method described in the Invention Patent Certificate No. TW I772201 B. Briefly, a dry powder of fruiting bodies of Cordyceps militaris (Bioray Biotech Co., Ltd.) was mixed with an ethanol solution having an ethanol concentration of 62.9% in a weight ratio of 1:10, followed by conducting a reflux extraction treatment at 65° C. for 88.3 minutes, so as to obtain a mixture. Next, the mixture was subjected to ultrasonication using an ultrasonic processor (Delta; Model: DC-100H) at a frequency of 40 KHz for 88.3 minutes, followed by filtration using a filter paper (ADVANTEC) with a pore size of 2.5 μm, thereby obtaining a filtrate. Afterward, the filtrate was dried by concentrating under reduced pressure, thereby obtaining the ethanol-extracted product of fruiting bodies of Cordyceps militaris.

2. Preparation of Ethanol-Extracted Product of Fruiting Bodies of Cordyceps cicadae:

For comparison purpose, an ethanol-extracted product of fruiting bodies of Cordyceps cicadae was prepared using procedures similar to those of the ethanol-extracted product of fruiting bodies of Cordyceps militaris as described in Section 1 of this example, except that a dry powder of fruiting bodies of Cordyceps cicadae (Bioray Biotech Co., Ltd.) was used to replace the dry powder of fruiting bodies of Cordyceps militaris.

### 3. Experimental Mice:

Female Bltw:CD1 (ICR) mice (hereinafter referred to as “ICR mice”) (10 weeks old, with a body weight of approximately 25 g to 28 g) used in this example were purchased from BioLASCO Taiwan Co., Ltd. All the experimental mice were housed in an animal room with an alternating 12-hour light and 12-hour dark cycle under standard temperature and humidity conditions. Furthermore, water and feed were provided ad libitum for all the experimental mice. All experimental procedures involving the experimental mice were approved by the Institutional Animal Care and Use Committee of Chung Shan Medical University and were in compliance with the Guide for the Care and Use of Laboratory Animals of Chung Shan Medical University.

### Experimental Procedures:

### A. Induction of Dry Eye Disease:

First, the ICR mice were randomly divided into five groups, namely, a normal control group (n=4), a pathological control group (n=5), two experimental groups (i.e., experimental groups 1 and 2) (n=5 in each group), and a comparative group (n=3). The mouse model of sleep deprivation (SD) used in this example was established according to the procedures described in Li S. et al. (2018), Exp. Mol. Med., 50:e451. In brief, each of the ICR mice in the pathological control group, the experimental groups 1 and 2, and the comparative group was subjected to a period of 16 sleep deprivation cycles each of which was performed for one day where a period of 19 hours to 20 hours of numerous of sleep deprivations conducted repeatedly was accompanied by a period of 4 hours to 5 hours rest. A respective one of the sleep deprivations in each sleep deprivation cycle was conducted as follows. Each of the ICR mice in the pathological control group, the experimental groups 1 and 2, and the comparative group was made to stand on a wooden stake (cross-sectional diameter: 1.5 cm) where a dish filled with water was disposed underneath, and when the ICR mouse fell asleep, it would fall into the water and hence woke up, and after that, the ICR mouse was placed on the wooden stake again. After completion of the 16 sleep deprivation cycles for a total period of 16 days, dry eye disease was successfully induced in the respective mouse. In addition, the ICR mice in the normal control group received no treatment.

B. Administration of Ethanol-Extracted Product of Fruiting Bodies of Cordyceps cicadae:

At the end of the 5^{th} day after induction of dry eye disease as described in section A of this example (i.e., after completion of 5 sleep deprivation cycles), each of the ICR mice in the experimental groups 1 and 2 was administered, via oral gavage, with the ethanol-extracted product of fruiting bodies of Cordyceps militaris obtained in Section 1 of the Experimental materials (dosage: 1 mg/kg body weight and 10 mg/kg body weight in experimental groups 1 and 2, respectively; prepared in 10% polyethylene glycol 400 (PEG 400) solution). Moreover, each of the ICR mice in the comparative group was administered, via oral gavage, with the ethanol-extracted product of fruiting bodies of Cordyceps cicadae obtained in Section 2 of the Experimental materials (dosage: 10 mg/kg body weight; prepared in 10% PEG 400 solution). As for the ICR mice in the normal control group and the pathological control group, only 10% PEG 400 solution was administered to each thereof. Each mouse was administered twice a day for a total period of ten days.

### C. Analysis of Tear Production:

At the end of the 15^{th} day after induction of dry eye disease as described in section A of this example (i.e., after completion of 15 sleep deprivation cycles), a Schirmer tear test strip with an area of 1 mm×10 mm was placed in a lower eyelid of each ICR mouse for 20 seconds. Thereafter, the Schirmer tear test strip was taken out from each ICR mouse, and the wetted length was determined and recorded.

The data thus obtained were analyzed according to the procedures as described in Section 1 of the General experimental procedures.

### D. Analysis of Tear Film Stability:

At the end of the 15^{th} day after induction of dry eye disease as described in section A of this example (i.e., after completion of 15 sleep deprivation cycles) and after completion of the analysis of tear production as described in Section C above, 3 μL of 5% fluorescein sodium (Sigma-Aldrich) was topically instilled into an ocular surface of each ICR mouse, followed by observation of the tear film morphology of the mouse eye using a slit-lamp biomicroscopy with cobalt blue light and then recordation of the tear film break-up time (TBUT).

The data thus obtained were analyzed according to the procedures as described in Section 1 of the General experimental procedures.

### E. Analysis of Tear Film Surface Regularity:

Prior to the start of the 16 sleep deprivation cycles to induce dry eye disease as described in section A of this example (i.e., on day 0) and at the end of the 16^{th} day after induction of dry eye disease as described in section A of this example (i.e., after completion of the 16 sleep deprivation cycles), the tear film morphology of each ICR mouse was observed using a corneal topographer and was photographed for 50 seconds, thereby obtaining an image of concentric rings which was projected onto a surface of the tear film. Subsequently, the concentric rings were subjected to observation. The higher the degree of distortion of the concentric rings is, the worse the tear film surface regularity is.

### F. Evaluation of Severity of Corneal Epithelial Injury:

At the end of the 16^{th} day after induction of dry eye disease as described in section A of this example (i.e., after completion of the 16 sleep deprivation cycles), 3 μL of 1% lissamine green (Sigma-Aldrich) was topically instilled into the ocular surface of each ICR mouse, followed by observation and photography using the slit-lamp biomicroscopy with cobalt blue light. Afterward, the corneal epithelial injury was assessed approximately according to the method described in Liou J. C. et al. (2015), Mol. Vis., 21:846-856. The degree of the corneal lissamine green staining was scored on a scale from 0.0 to 0.4. The higher the score is, the severer the corneal epithelia injury is.

The data thus obtained were analyzed according to the procedures as described in Section 1 of the General experimental procedures.

### Result:

### A. Analysis of Tear Production:

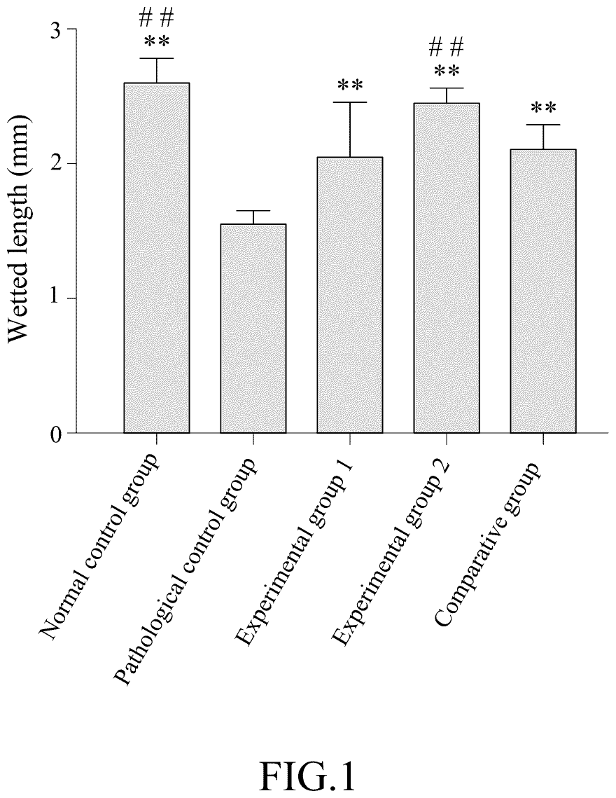

Referring to FIG. 1, compared with the normal control group, the wetted length determined in the pathological control group had a significant reduction, indicating that sleep deprivation indeed induced dry eye disease and caused insufficient tear production in the ICR mice of the pathological control group. In comparison with the pathological control group, the wetted length determined in each of the experimental groups 1 and 2 and the comparative group had a significant increase. In addition, the wetted length determined in the experimental group 1 showed no obvious difference compared with that of the comparative group, and the wetted length determined in the experimental group 2 had a significant increase. These results show that the ethanol-extracted product of fruiting bodies of Cordyceps militaris can effectively alleviate the deficiency of tear production in the ICR mice with sleep deprivation-induced dry eye disease, and such efficacy is better than that of the ethanol-extracted product of fruiting bodies of Cordyceps cicadae under the same dose.

### B. Analysis of Tear Film Stability:

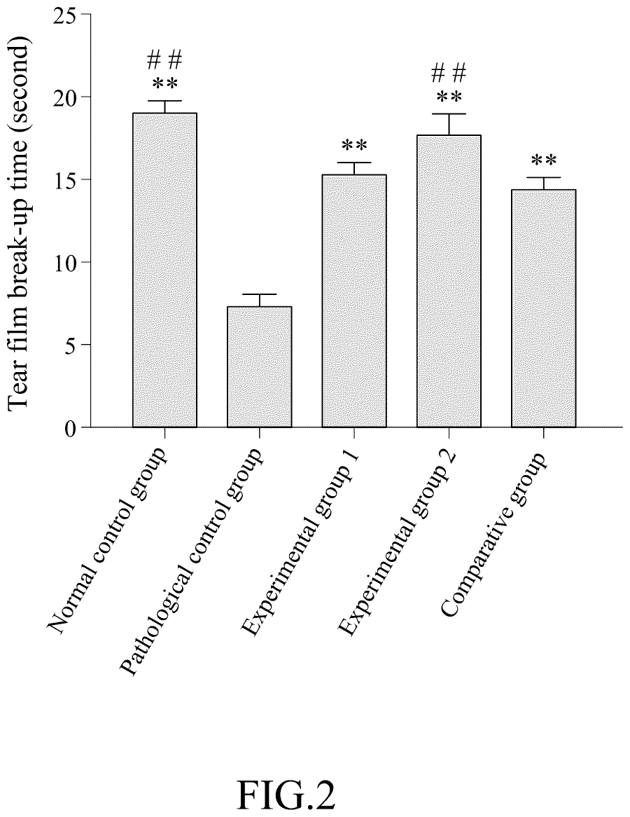

Referring to FIG. 2, compared with the normal control group, the tear film break-up time recorded in the pathological control group was significantly reduced, indicating that sleep deprivation indeed induced dry eye disease and caused tear film instability in the ICR mice of the pathological control group. In comparison with the pathological control group, the tear film break-up time recorded in each of the experimental groups 1 and 2 and the comparative group was significantly increased. In addition, the tear break-up time recorded in the experimental group 1 showed no obvious difference compared with that of the comparative group, and the tear break-up time recorded in the experimental group 2 was significantly increased. These results show that the ethanol-extracted product of fruiting bodies of Cordyceps militaris can effectively enhance tear film stability, and such efficacy is better than that of the ethanol-extracted product of fruiting bodies of Cordyceps cicadae under the same dose.

### C. Analysis of Tear Film Surface Regularity:

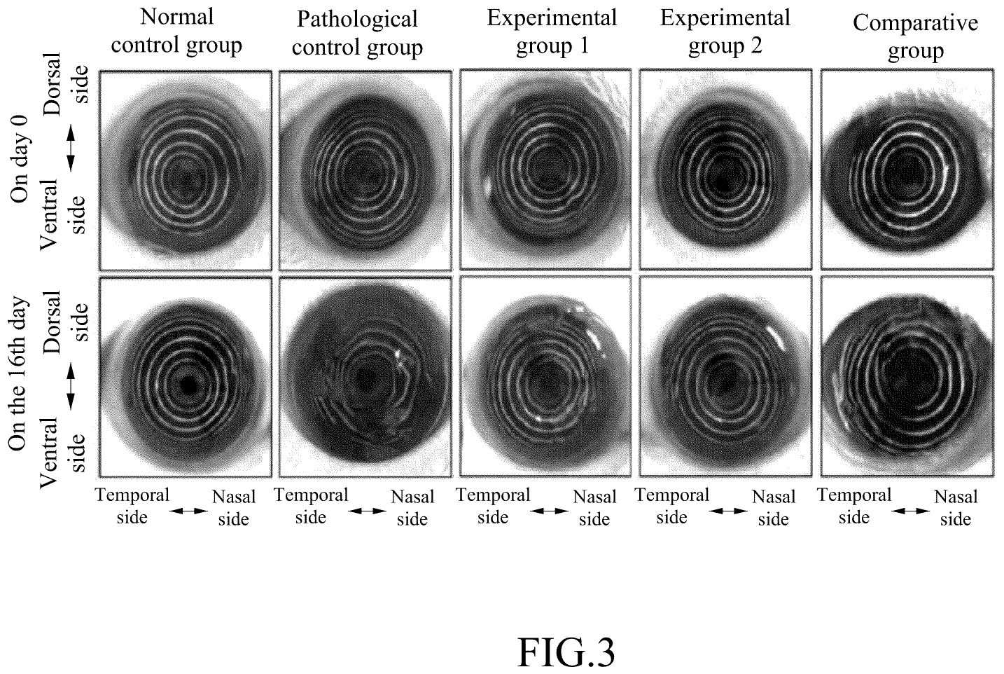

Referring to FIG. 3, after completion of the 16 sleep deprivation cycles, the concentric rings observed in the pathological control group showed obvious distortion compared with those observed in the normal control group, indicating that sleep deprivation indeed induced dry eye disease and caused tear film surface irregularity in the ICR mice of the pathological control group. In comparison with the pathological control group, the degree of distortion of the concentric rings observed in each of the experimental groups 1 and 2 was significantly reduced, and the degree of distortion of the concentric rings observed in the comparative group was only slightly reduced. These results show that the ethanol-extracted product of fruiting bodies of Cordyceps militaris, even at a low dose, can effectively improve tear film surface regularity, and such efficacy is better than that of the ethanol-extracted product of fruiting bodies of Cordyceps cicadae.

### D. Evaluation of Severity of Corneal Epithelial Injury:

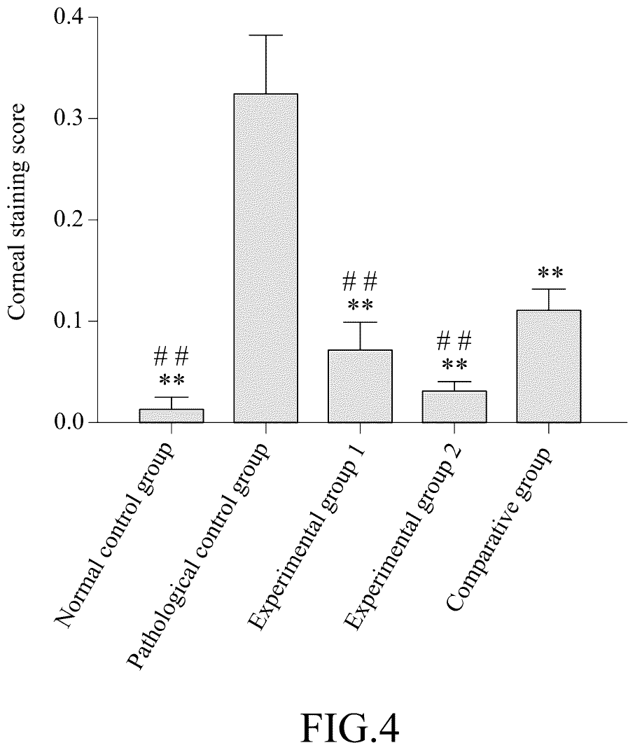

Referring to FIG. 4, compared with the normal control group, the corneal staining score determined in the pathological control group was significantly increased, indicating that sleep deprivation indeed induced dry eye disease and caused corneal epithelial injury in the ICR mice of the pathological control group. In comparison with the pathological control group, the corneal staining score determined in each of the experimental groups 1 and 2 and the comparative group was significantly reduced. In addition, compared with the comparative group, the corneal staining score determined in each of the experimental groups 1 and 2 was significantly reduced. These results show that the ethanol-extracted product of fruiting bodies of Cordyceps militaris, even at a low dose, can effectively alleviate the severity of the corneal epithelial injury, and such efficacy is better than that of the ethanol-extracted product of fruiting bodies of Cordyceps cicadae.

In sum, the ethanol-extracted product of fruiting bodies of Cordyceps militaris according to the disclosure can effectively alleviate the deficiency of tear production, improve tear film surface regularity, enhance tear film stability, and also alleviate the severity of the corneal epithelial injury, and hence is capable of alleviating dry eye disease. Therefore, the ethanol-extracted product of fruiting bodies of Cordyceps militaris according to the disclosure is expected to have a high potential to be used as a therapeutic agent for alleviating dry eye disease.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for alleviating dry eye disease, comprising administering to a subject in need thereof a pharmaceutical composition containing an ethanol-extracted product of fruiting bodies of Cordyceps militaris.

2. The method as claimed in claim 1, wherein the dry eye disease is caused by a factor selected from the group consisting of a sleep disorder, aging, Sjögren's syndrome, a particulate matter (PM), and combinations thereof.

3. The method as claimed in claim 2, wherein the dry eye disease is caused by the sleep disorder.

4. The method as claimed in claim 1, wherein the pharmaceutical composition is in a dosage form for oral administration.
